(19) United States
(12) Patent Application Publication
(10) Pub. No.: US 2026/0315073 A1
(43) Pub. Date: Oct. 15, 2026

(54) INTRATUMORAL OCCLUSION INSTRUMENT

(71) Applicant: MICROPORT NEUROTECH (SHANGHAI) CO., LTD., Shanghai (CN)

(72) Inventors: Shuang GUO, Shanghai (CN); Mengqi CHANG, Shanghai (CN); Bing CHEN, Shanghai (CN); Yuanyuan ZHANG, Shanghai (CN)

(21) Appl. No.: 19/137,691

(22) PCT Filed: Oct. 31, 2023
(86) PCT No.: PCT/CN2023/128118

§ 371 Date: Jun. 11, 2025

(30) Foreign Application Priority Data
Dec. 15, 2022 (CN) 202211613620.3

(51) Int. Cl. A61B 17/12

(52) U.S. Cl. CPC A61B 17/12113; A61B 17/12172

(57) ABSTRACT

An intratumoral occlusion device switchable between a collapsed configuration and an expanded configuration includes a mesh-like body structure and a proximal fixing structure. The mesh-like body structure includes an inner support structure and an outer occlusion structure and is formed from a braided mesh tube. The inner support structure is formed by inwardly folding one portion of the braided mesh tube into the interior of another portion of the braided mesh tube, and the outer occlusion structure is formed by the another portion of the braided mesh tube that is unfolded. A proximal end of the outer occlusion structure is connected to the proximal fixing structure. The inner support structure proximally extends toward and is connected to the proximal fixing structure.

### TECHNICAL FIELD

The present invention relates to the field of medical devices, particularly to an intratumoral occlusion device for aneurysm filling.

### BACKGROUND

As one type of cerebrovascular disease commonly found in clinical practice, intracranial aneurysms are associated with high mortality and disability rates. According to a non-invasive imaging study, 7% of the Chinese population is with intracranial aneurysms. Among those with subarachnoid hemorrhages caused by ruptured intracranial aneurysms, up to 45% died within 30 days, and about 30% of the survivors are suffering from various degrees of neurological impairment.

In recent years, vascular intervention has become a mainstream therapy for intracranial aneurysms thanks to its advantages including direct lesion access without affecting brain tissue and minimal surgical trauma. At present, vascular intervention for intracranial aneurysms is accomplished essentially by coil embolization or flow diversion. Coil embolization involves filling an aneurysm with a preformed coil released into the aneurysm from a catheter. The coil causes stagnation of blood flow in the aneurysm lumen, creating a clot over time, which blocks additional blood from entering the aneurysm. In this way, further expansion of the aneurysm is prevented. Flow diverters are used to treat aneurysms by vascular reconstruction, and exhibit heightened long-term efficacy particularly against large and giant aneurysms. However, for some large aneurysms, the simple use of a flow diverter for treatment is associated with a risk of later rupture. There are also some novel devices available for intra-aneurysmal embolization (e.g., WEB, Artisse and Contour devices), which are typically made of shape memory alloy materials and pre-formed in particular shapes. Such a device is designed to be delivered through a catheter into an aneurysm, where it is pushed out of a sheath and then self-expands to recover its pre-formed shape, thus closing the aneurysm. However, the above approaches are associated with the problems as follows:
1) Coil embolization of a wide-neck and bifurcation aneurysm requires the implantation of an additional device (e.g., an assist stent, or a flow diverter) for preventing herniation of the coil. However, the combined use of the multiple devices increases the surgical time, therapeutic cost and likelihood of adverse events, leading to low efficiency and imposing stringent requirements on surgeons' dexterity and experience. On the other hand, the use of only the coil is associated with a risk of herniation from the aneurysm. Further, the combined use of other devices is associated with an increased risk of ischemic complications.
2) Flow diverters are not suitable for the treatment of bifurcation aneurysms and aneurysms in arteries with perforating branches due to a risk of vascular occlusion. In addition, patients implanted with flow diverters have to rely on long-term dual antiplatelet therapy and suffer from a risk of hemorrhagic complications after surgery.
3) The novel intratumoral embolization devices are also each associated with a number of problems. WEB devices are mainly used to treat wide-neck bifurcation aneurysms, in particular regular aneurysms. The device has a relatively long deployment length, and its distal anchoring points may exert impact on the aneurysm wall. Moreover, the single spherical or cylindrical structure provides limited conformity to the aneurysm wall. After long-term implantation, it may become compressed and displaced, potentially leading to aneurysm recurrence. Artisse devices operate in a similar way as WEB devices. Although they are absent of a distal anchoring point, they can achieve a good embolization effect only when their spherical geometry can conform well to the aneurysm wall. Contour devices are disc-shaped and suited to the treatment of bifurcation or basilar apex aneurysms. They typically require repeated relocation to completely cover the aneurysm neck. Otherwise, their stability in the aneurysm will be affected.

It should be noted that the information disclosed in this Background section is merely intended to provide a better understanding of the general context of the present invention and should not be taken as an acknowledgement or any form of admission that the information forms part of the common general knowledge of those skilled in the art.

### SUMMARY OF THE INVENTION

It is an object of the present invention to provide an intratumoral occlusion device, which overcomes at least one of the above described problems with conventional vascular treatments.

To this end, the present invention provides an intratumoral occlusion device switchable between a collapsed configuration and an expanded configuration. The intratumoral occlusion device comprises a mesh-like body structure and a proximal fixing structure, and the mesh-like body structure comprises an inner support structure and an outer occlusion structure and is made from a braided mesh tube. The inner support structure is formed by inwardly folding one portion of the braided mesh tube into the interior of another portion of the braided mesh tube, and the outer occlusion structure is formed by the another portion of the braided mesh tube that is un. The outer occlusion structure is connected at a proximal end thereof to the proximal fixing structure, and the inner support structure proximally extends toward and is connected to the proximal fixing structure.

Optionally, in the expanded configuration, a radial diameter of the inner support structure may increase from both proximal and distal ends to the middle portion of the inner support structure. Alternatively, in the expanded configuration, a radial diameter of the inner support structure may decrease from a distal end to a proximal end of the inner support structure.

Optionally, in the expanded configuration, the outer contour of the inner support structure may be a fusiform or inverted conical.

Optionally, in the expanded configuration, a maximum radial diameter of the inner support structure may not be greater than ½ of a radial diameter of the outer occlusion structure.

Optionally, in the expanded configuration, an axial height of the outer occlusion structure may not be greater than a radial diameter of the outer occlusion structure and not be less than ½ of a radial diameter of the outer occlusion structure.

Optionally, in the expanded configuration, the axial height of the outer occlusion structure may be less than the radial diameter of the outer occlusion structure.

Optionally, in the expanded configuration, outer contour of the outer occlusion structure may have a shape of a sphere or cylinder.

Optionally, in the expanded configuration, the axial height of the outer occlusion structure may be 0.6-0.8 times the radial diameter of the outer occlusion structure.

Optionally, in the expanded configuration, an axial height of the inner support structure may not be greater than an axial height of the outer occlusion structure. Alternatively or additionally, in the expanded configuration, the axial height of the inner support structure may not be greater than a radial diameter of the outer occlusion structure.

Optionally, in the expanded configuration, an axial height of the inner support structure may not be less than ⅔ of an axial height of the outer occlusion structure. Alternatively or additionally, in the expanded configuration, the axial height of the inner support structure may not be less than ⅔ of a radial diameter of the outer occlusion structure.

Optionally, mesh openings in a proximal end face of the outer occlusion structure may have an area not exceeding 0.1 mm².

Optionally, the intratumoral occlusion device may comprise a proximal end and a distal end, wherein at the distal end, the braided mesh tube smoothly transits at the inwardly folded location to form a distal opening.

Optionally, in the expanded configuration, a diameter of the distal opening may be not less than ⅓ of a radial diameter of the outer occlusion structure.

The present invention provides an intratumoral occlusion device comprising a mesh-like body structure and a proximal fixing structure. The mesh-like body structure comprises an inner support structure and an outer occlusion structure, and is made from a braided mesh tube. The inner support structure is formed by inwardly folding one portion of the braided mesh tube into the interior of another portion of the braided mesh tube, and the outer occlusion structure is formed by the another portion of the braided mesh tube that is unfold. A proximal end of the outer occlusion structure is connected to the proximal fixing structure, and a proximal end of the inner support structure extends toward and is connected to the proximal fixing structure. This single-anchor design of the intratumoral occlusion device achieved simply by inwardly folding the braided mesh tube allows it to have a shorter axial length after release and a more compliant and smoother distal end which reduces its impact on the dome of an aneurysm in an initial phase of the release process and hence the risk of causing rupture of the aneurysm. In addition, the inner support structure formed by the inwardly folding imparts enhanced axial support performance to the intratumoral occlusion device and ensures its long-term stability when it is being filled in an aneurysm.

### BRIEF DESCRIPTION OF THE DRAWINGS

The drawings accompanying the present invention are not necessarily drawn to scale, and some of the components and structures in the drawings may be exaggerated in size for the sake of clarity. Many variants of the illustrated embodiments can be contemplated. Accordingly, the illustration of the various aspects and elements of the embodiments shown in the drawings is not intended to limit the scope of the invention in any sense, in which:

FIG. 1 is a schematic front view of an intratumoral occlusion device in an expanded configuration according to a first embodiment of the present invention;

FIG. 2 schematically illustrates the intratumoral occlusion device according to the first embodiment of the present invention, which is being released;

FIG. 3 schematically illustrates the intratumoral occlusion device according to the first embodiment of the present invention, which has been completely released into an aneurysm;

FIG. 4 is a schematic front view of an intratumoral occlusion device in an expanded configuration according to a second embodiment of the present invention; and

FIG. 5 is a schematic front view of an intratumoral occlusion device in an expanded configuration according to a third embodiment of the present invention.

### LIST OF REFERENCE NUMERALS

10, 20, 30—intratumoral occlusion device; 11—mesh-like body structure; 111—inner support structure; 112—outer occlusion structure; 12—proximal fixing structure; 10A—proximal end of intratumoral occlusion device; 10B—distal end of intratumoral occlusion device; 10C—distal opening; D1—radial diameter of inner support structure; H1—axial height of inner support structure; D2—radial diameter of outer occlusion structure; H2—axial height of outer occlusion structure; 1—microcatheter; 2—aneurysm; 21—aneurysm neck opening; 3—pushrod.

### DETAILED DESCRIPTION

Various exemplary embodiments are described below. Reference is made to these examples in a non-limiting sense, as it should be noted that they are provided to illustrate more broadly applicable aspects of the devices, systems and methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present invention. All such modifications are intended to be within the scope of the claims made herein.

Any dimensions mentioned in the foregoing general description or the following detailed description are exemplary only, and should not be taken as limiting the subject matter of the present invention, unless so indicated in various exemplary embodiments. In addition, the various configurations of the embodiments described herein are mutually supplementary, but not purely alternative, unless so stated. In other words, a configuration of one embodiment can be freely combined with configuration(s) of other embodiment(s), as would be readily understood by those of ordinary skill in the art, unless they are explicitly described as being alternative to each other or one another.

As used herein, the terms “proximal end” and “distal end” describe relative orientations, positions and directions of components of a medical device or actions thereon, as viewed by an operator operating the medical device. Without wishing to be limiting, “proximal end” usually refers to an end of the medical device closer to the operator, and “distal end” to an end that enters the body of a patient first, during normal operation of the device. In the context herein, “distal end” and “proximal end” refer to relative locations, rather than particular end portions. For example, a “distal end” of a mesh-like structure refers to a location near the distal end of the mesh structure, rather than exactly the distal end.

As used herein to describe an intratumoral occlusion device being filled in an aneurysm, “axial” usually refers to a heightwise direction of the device from an aneurysm neck opening to the aneurysm dome, and “radial” refers to a diametrical direction of the device along a diameter of the aneurysm neck opening. “Expanded configuration” includes a “freely expanding configuration”, in which the device is not under stress, and a “filled configuration”, in which it is filled in the aneurysm and compressed by the aneurysm wall. “Radial diameter” refers to the outer diameter of a projected contour formed by projection of an outer contour onto a cross-sectional plane perpendicular to the axial direction.

Before describing the present invention in detail, it is understood that conventional aneurysm closure devices are single-layer dense meshes typically in the shape of a ball or cylinder and designed to be filled in cavity of the aneurysms. Such a single-layer dense mesh is absent of internal axial support and, after being filled in an aneurysm, is prone to axial contraction due to impingement of blood flow over time. When this happens, the aneurysm neck will be incompletely covered, or the aneurysm may recur. In order to overcome this, the present application seeks to provide an intratumoral occlusion device essentially used to occlude an aneurysm, in particular an intracranial aneurysm. It is particularly suitable for the treatment of saccular wide-necked aneurysms. The intratumoral occlusion device of present application is a continuous double-layer mesh-like structure, and provides an inner support structure in the outer occlusion structure. The inner support structure can greatly increase axial support performance of the device and ensures stable long-term filling. In particular, the inner support structure may have a fusiform or inverted conical shape, which imparts even better axial support performance.

The present application will be further described below with reference to preferred embodiments thereof. Should there be no conflict, the embodiments described hereunder and features thereof can complement or be combined with each other or one another.

### Embodiment 1

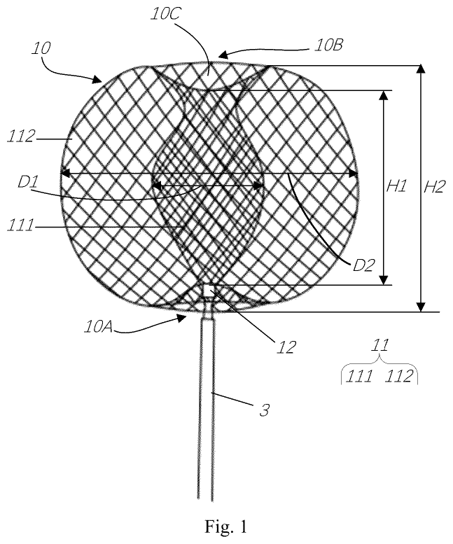

Referring to FIGS. 1 to 3, in a first embodiment of the present application, there is provided an intratumoral occlusion device 10 including a mesh-like body structure 11 and a proximal fixing structure 12. The mesh-like body structure 11 comprises an inner support structure 111 and an outer occlusion structure 112. The mesh-like body structure 11 is a continuous double-layer mesh structure made from a braided mesh tube. In particular, one portion of the braided mesh tube is inwardly folded into the interior of the rest and thereby forms the inner support structure 111, with the unfolded portion of the braided mesh tube forming the outer occlusion structure 112. The outer occlusion structure 112 provides full contact with the inner wall of an aneurysm and substantially fills the entire lumen of the aneurysm.

The intratumoral occlusion device 10 includes a proximal end 10A and a distal end 10B. A distal opening 10C is formed at the distal end 10B as a result of the inward folding of the braided mesh tube. It will be understood that the braided mesh tube folds inwardly in a smooth arc without forming distinct fold line, resulting in a rounded transition at the inward folding position and forming a relatively large distal opening 10C. This imparts desirable compliance to the intratumoral occlusion device 10, minimizing any possible damage that it causes to the aneurysm dome when coming into contact therewith. Therefore, the distal transition between the outer occlusion structure 112 and the inner support structure 111 is rounded and smooth, ensuring desirable axial support performance while taking into account compliance of the device itself and enabling sheathing and delivery of the intratumoral occlusion device 10. Further, in an expanded configuration, the distal opening 10C has a diameter not less than ⅓ of a radial diameter D2 of the outer occlusion structure 112.

The proximal end of the outer occlusion structure 112 is connected to the proximal fixing structure 12. Specifically, the proximal end of the inner support structure 111 extends towards the proximal fixing structure 12 and is connected to the proximal fixing structure 12. Opposite ends of the braided mesh tube before folding form respective proximal ends of the inner support structure 111 and the outer occlusion structure 112. Braid wire ends at the proximal ends of the inner support structure 111 and the outer occlusion structure 112 are secured and fixed by the proximal fixing structure 12. The proximal fixing structure 12 may be radiopaque structure, or not, for example, in the form of a radiopaque sleeve. This radiopaque structure may be made of platinum, gold, tantalum, or an alloy of these and/or other radiopaque materials.

It will be understood that the intratumoral occlusion device 10 of this embodiment has a single proximal anchor, i.e., the location to which braid wire ends at the opposite ends of the braided mesh tube are secured and fixed. In other words, all the braid wire ends are gathered at the proximal end 10A of the intratumoral occlusion device 10, and secured and fixed by the proximal fixing structure 12. Additionally, the distal end 10B of the intratumoral occlusion device 10 is recessed and absent of any projecting anchor. Compared with having two anchors respectively at the distal and proximal ends, providing a single anchor at the proximal end enables the intratumoral occlusion device 10 to have a reduced axial length after release and to be more compliant and smooth at the distal end. The intratumoral occlusion device 10 will self-expand as it is pushed out of a microcatheter 1, reducing an impact that may be otherwise exerted on the aneurysm dome in an initial phase of the release process, and reducing the aneurysm rupture risk. In addition, the intratumoral occlusion device 10 is easier to release, making the entire procedure simper, lowering the requirements on the operator's personal experience in aneurysm embolization shortening the surgical time and increasing surgical efficiency.

In order to achieve the single-proximal-anchor device, the applicant has attempted to interweave and braid wires and gather their ends at the proximal end to form a proximal anchor. The resulting structure is overall a braided mesh, except for the proximal anchor. Despite good compliance, this simply braided structure could not provide sufficient support performance and would be prone to axial contraction when filled in an aneurysm, which makes it impossible to cover an aneurysm neck opening 21. In addition, the braiding is complex, leading to difficult fabrication. In view of this, it is proposed herein to simply inwardly folding a braided mesh tube and securing and fixing braid wire ends at opposite ends thereof by a proximal fixing structure 12. Finally, the resulting structure is subject to heat setting, resulting in an intratumoral occlusion device 10 having an inner support structure 111 and an outer occlusion structure 112. This fabrication process is easier to implement, and a single proximal anchor can be obtained while taking into account overall compliance and support performance of the intratumoral occlusion device 10, which enables the device to better geometrically conform to the inner wall of an aneurysm and stably cover an aneurysm neck opening 21 for a long time, reducing the risk of recurrence.

The applicant has also attempted to outwardly fold one portion of a braided mesh tube over the rest thereof. However, this tends to impair the performance of the braided mesh tube and reduce the coverage of its material. In contrast, inwardly folding one portion of a braided mesh tube into the interior of the another of braided mesh tube to form an inner support structure 111, as proposed herein, does not affect the braided mesh tube considerably, and the overall folding process is controllable so that the mesh opening size after folding remains consistent with the mesh opening size before folding, maintaining the dense mesh performance of the mesh-like body structure 11.

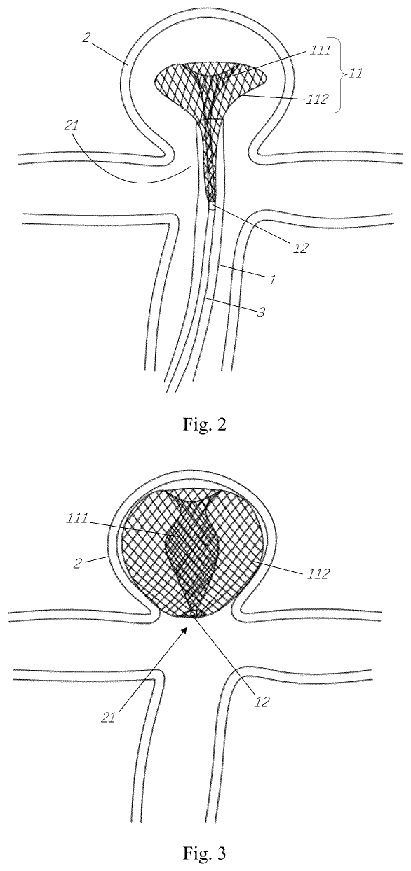

In general terms, the intratumoral occlusion device 10 has an expanded configuration and a collapsed configuration and is switchable between these configurations. As shown in FIG. 2, the intratumoral occlusion device 10 can be delivered through a microcatheter 1. It can be crimped in the collapsed configuration within the microcatheter 1, and after being pushed out of the microcatheter 1, restore a freely expanding configuration. As shown in FIG. 3, the intratumoral occlusion device 10 can also transition from the freely expanding configuration to a filled configuration, in which it is filled in an aneurysm 2.

As shown in FIGS. 1 and 2, the intratumoral occlusion device 10 is detachably coupled to a distal end of a pushrod 3 via the proximal fixing structure 12, and it can be pushed by the pushrod 3 to move within the microcatheter 1 along an axis thereof. The present application is not limited to any particular method for coupling the proximal fixing structure 12 to the distal end of the pushrod 3, as long as the coupled two can be successfully detached and separated from each other. For example, the detachment can be accomplished electrochemically, by heat melting, mechanically or hydrolytically.

The intratumoral occlusion device 10 may be implanted by an interventional procedure, in which the microcatheter 1 is first inserted into an aneurysm 2 in a parent vessel and the entire intratumoral occlusion device 10 is then advanced in the microcatheter 1 until its distal end reaches an aneurysm neck opening 21 of an aneurysm 2. After that, with the microcatheter 1 being held stationary, the pushrod 3 is pushed forward to gradually urge the intratumoral occlusion device 10 into the aneurysm 2. In this process, the intratumoral occlusion device 10 continues self-expanding. As can be seen from FIG. 2, during release, the distal end of the outer occlusion structure 112 is first pushed out of the microcatheter 1, placing the device into a partially released configuration. Since the distal end of the outer occlusion structure 112 is compliant and has a large area, it causes only minimal damage to the aneurysm dome. Additionally, compared with devices having two anchors respectively at distal and proximal ends, this single-anchor device has a smaller length after release and is easier to manipulate. Once the entire intratumoral occlusion device 10 is pushed out from the microcatheter 1, it is completely released into the aneurysm 2. As the intratumoral occlusion device 10 is completely received in the aneurysm 2, long-term dual antiplatelet therapy is not necessary, reducing the risk of hemorrhagic complications.

In the embodiment of FIG. 3, after the intratumoral occlusion device 10 is completely released into an aneurysm 2, it substantially fully fills the aneurysm lumen. At this point, a proximal end face of the outer occlusion structure 112 effectively covers the aneurysm neck opening 21, reducing the impact of blood upon the aneurysm lumen. Moreover, it provides a scaffold capable of facilitating healing of the aneurysm neck. The rest of the outer occlusion structure 112 conforms to the inner wall of the aneurysm. The outer occlusion structure 112 interacts with the wall of the aneurysm to enable stable filling of the intratumoral occlusion device 10, and under support of the inner support structure 111, the axial stability of the intratumoral occlusion device 10 for a long time can be maintained, avoiding recurrence of the aneurysm after surgery. In particular, the inner support structure 111 imparts significantly enhanced axial support performance to the single-proximal-anchor intratumoral occlusion device 10, reduces its axial shrinkage and effectively mitigates the impact of the “water-hammer” effect of blood on the intratumoral occlusion device 10 over time, provides long-term resistance to blood flow-induced compression. Thus, stable long-time filling of the aneurysm by the intratumoral occlusion device 10 is ensured.

As shown in FIG. 1, in the expanded configuration, an axial height H2 of the outer occlusion structure 112 is not greater than its radial diameter D2. This allows for a small length after release, which results in a reduced impact on the aneurysm dome. Additionally, the axial height H2 of the outer occlusion structure 112 is not less than ½ of its radial diameter D2 in the expanded configuration. That is, the axial height H2 of the outer occlusion structure 112 is 50%-100% of the radial diameter D2 in the expanded configuration. This ensures that the intratumoral occlusion device 10 can substantially fully fill the aneurysm lumen. When fully filling the aneurysm lumen, the intratumoral occlusion device 10 can provide support to the inner wall of the aneurysm, preventing its rupture. Failure in full filling of the aneurysm lumen will give rise to a risk of bleeding or rebleeding at a later time. Further, when the intratumoral occlusion device 10 fully fills the aneurysm lumen, the inner support structure 111 is enabled to provide more effective axial support to the intratumoral occlusion device 10.

Preferably, in the expanded configuration, the axial height H2 of the outer occlusion structure 112 is less than its radial diameter D2 in the expanded configuration. This reduced axial height allows the intratumoral occlusion device 10 to have a smaller length after release, allowing for easier operation of the operator, reducing the impact on the aneurysm dome that is prone to rupture and lowering the risk of rupture during the perioperative period. Further, when the axial height H2 of the outer occlusion structure 112 is less than its radial diameter D2 in the expanded configuration, the intratumoral occlusion device 10 no longer needs to match the shape of the entire lumen of any particular aneurysm and can be suitably used to treat a wider range of aneurysms, such as regular and irregular aneurysms.

Referring to FIGS. 1 and 3, in this embodiment, when in the expanded configuration, the outer occlusion structure 112 has a spherical outer contour, and the axial height H2 of the outer occlusion structure 112 is substantially equal to the radial diameter D2, enabling it to almost fully fill the entire aneurysm lumen.

The radial diameter D2 of the outer occlusion structure 112 in the expanded configuration is essentially determined by the size of an intended aneurysm. Generally, the radial diameter D2 of the expanded outer occlusion structure 112 may be determined by the size of common aneurysms. Optionally, the radial diameter D2 of the expanded outer occlusion structure 112 may range from 3 mm to 25 mm. This range substantially addresses most common aneurysms.

As to disturb flow, the dense mesh of the outer occlusion structure 112 is desired to have appropriate mesh openings, which should not be excessively large. Preferably, mesh openings in the proximal end face of the outer occlusion structure 112 have an area not larger than 0.1 mm². This can reduce the impact of blood flow upon the aneurysm lumen, while enabling the mesh to serve as a scaffold, onto which the endothelial cells can grow and spread, facilitating healing of the aneurysm neck.

The present application is not limited to any particular material, from which the braided mesh tube is fabricated. Both elastic metal materials and macromolecular materials with good elasticity are possible. Preferably, the braided mesh tube is formed from braid wires made of a material with shape memory properties and thus exhibits good compliance. Examples of the material with shape memory properties may include shape memory alloy materials, such as nickel-titanium (Ni—Ti) alloys and nickel-titanium-cobalt (Ni—Ti—Co) alloy. For example, the wires may be double-layer composite metal wires (e.g., Ni—Ti@Pt). Alternatively, the wires of the braided mesh tube may be made of a polymeric material with certain shape recovery capabilities, such as poly-p-dioxanone (PDO), poly(L-lactide-co-ε-caprolactone) (PLC), polyurethane (PU), amorphous polynorbornene or the like. Still alternatively, the braided mesh tube may be formed from both shape memory alloy and radiopaque metal (e.g., Pt, Pt—Ir, etc.) wires, or from composite wires made of both shape memory alloy and radiopaque materials (e.g., DFT wires). The braided mesh tube may be formed from braid wires made of either the same or different materials.

Further, the braided mesh tube may be formed from 48-144 wires with a diameter of 0.0005-0.002 inches. This enables the braided mesh tube to exhibit good compliance and support performance.

Preferably, the inner support structure 111 also shows a certain degree of compliance, which allows the inner support structure 111 to have an axial contraction margin and increases the overall compliance of the intratumoral occlusion device 10. To this end, the inner support structure 111 should not be too soft or stiff. If the inner support structure 111 is excessively stiff, despite satisfactory axial support performance, the overall compliance of the intratumoral occlusion device 10 will be affected, making it impossible to geometrically conform to the inner wall of an aneurysm. On the other hand, if the inner support structure 111 is too soft, it would not able to provide sufficient axial support to the intratumoral occlusion device 10, making it prone to axial contraction. In view of this, according to the present invention, the inner support structure 111 is designed as a mesh structure, which is axially contactable to a certain extent. To this end, the inner support structure 111 is designed to have a non-constant radial diameter D1 in the expanded configuration. That is, instead of a hollow mesh tube with a constant radial diameter, the inner support structure 111 is designed to have a varying radial diameter D1 in the expanded configuration.

More specifically, in the expanded configuration, the radial diameter D1 of the inner support structure 111 increases from both the proximal and distal ends to the middle thereof, making the expanded inner support structure 111 generally appear fusiform. Alternatively, the radial diameter D1 of the inner support structure 111 deceases from the distal end to the proximal end thereof in the expanded configuration, making the expanded inner support structure 111 generally appear inverted conical. Compared with a spherical shape, both the fusiform and inverted conical shapes enable effectively enhanced axial support performance.

As shown in FIG. 1, in this embodiment, the expanded inner support structure 111 has a fusiform outer contour. In this case, the inner support structure 111 is implemented as a fusiform mesh structure broad in the middle and narrow at both ends. The fusiform inner support structure 111 ensures good axial support performance while exhibiting a certain degree of compliance. This results in a greater axial deformation margin upon radial contraction of the outer occlusion structure 112.

In the expanded configuration, the inner support structure 111 has an axial height H1 not greater than the axial height H2 of the outer occlusion structure 112 and/or the radial diameter D2 of the outer occlusion structure 112. More preferably, in the expanded configuration, the axial height H1 of the inner support structure 111 is not less than ⅔ of the axial height H2 of the outer occlusion structure 112. For example, the former may be equal to ⅔, ¾ or ⅚ of the latter. Alternatively or additionally, in the expanded configuration, the axial height H1 of the inner support structure 111 is not less than ⅔ of the radial diameter D2 of the outer occlusion structure 112. For example, the former may be equal to ⅔, ¾ or ⅚ of the latter. Through configuring the axial height H1 of the expanded inner support structure 111 in this way, the intratumoral occlusion device 10 can be avoided from encountering increased resistance during delivery due to an excessively large recess, as well as from, as much as possible, difficult sheathing due to the presence of recesses at both the distal and proximal ends of the outer occlusion structure 112.

Preferably, in the expanded configuration, a maximum radial diameter of the inner support structure 111 is not greater than ½ of the radial diameter D2 of the outer occlusion structure 112. For example, the former may be equal to ¼, ⅓ or ½ of the latter. This can avoid an unnecessarily large axial contraction margin of the intratumoral occlusion device 10 due to excessive bulging of the inner support structure 111, which reduces its axial support performance. It should be understood that a greater radial diameter of the inner support structure 111 imparts better axial compliance, but an excessively large radial diameter of the inner support structure 111 is detrimental to the axial support performance.

As shown in FIGS. 1 to 3, as an example, in the expanded configuration, the outer occlusion structure 112 has a spherical outer contour and the inner support structure 111 has a fusiform outer contour. Moreover, in the expanded configuration, the axial height H1 of the inner support structure 111 is ¾ of the radial diameter D2 of the outer occlusion structure 112, and the maximum radial diameter of the inner support structure 111 is ⅓ of the radial diameter D2 of the outer occlusion structure 112. This arrangement can minimize the impact of the intratumoral occlusion device 10 on the dome of an aneurysm in an initial phase of the release process and allows the device to have a short length after release, which facilitates surgical operation, enables stable filling and imparts desirable long-term axial stability.

### Embodiment 2

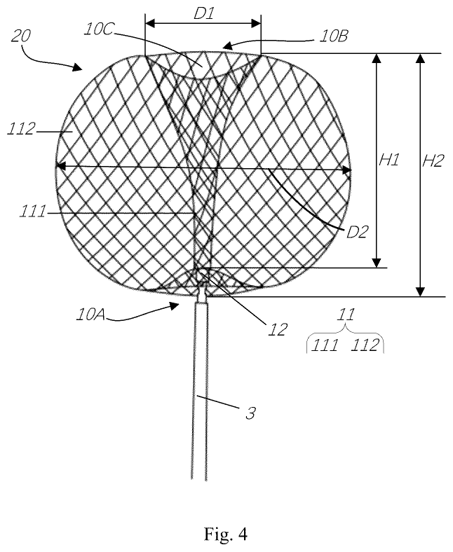

FIG. 4 shows an intratumoral occlusion device 20 according to a second embodiment of the present application. Corresponding structures in the intratumoral occlusion device 20 of this embodiment have been given the same reference numerals as structures in the first embodiment. The intratumoral occlusion device 20 of this embodiment is structured substantially in the same way as the intratumoral occlusion device 10 of the first embodiment, and features common to the two embodiments are not repeated here. From the following description, those skilled in the art will appreciate how the second embodiment differs from the first embodiment.

In the exemplary embodiment of FIG. 4, in the expanded configuration, the outer occlusion structure 112 has a spherical outer contour and the inner support structure 111 has an inverted conical outer contour. Here, by “inverted conical”, it is intended to mean that the expanded inner support structure 111 has a radial diameter D1 decreasing from the distal end to the proximal end. The radial diameter D1 may gradually decrease across the entire length, or first gradually decrease across a portion and then remains the same across the rest. Compared with the fusiform shape, the inverted conical shape enables the intratumoral occlusion device 20 to encounter even less resistance during retrieval. Specifically, compared with the case of the fusiform inner support structure 111, the intratumoral occlusion device 20 with the inverted conical inner support structure 111 will encounter less resistance during its retrieval into a microcatheter 1 and has a smaller axial extension margin, which imparts even better axial support performance.

As an example, in the expanded configuration, the axial height H1 of the inner support structure 111 is ⅚ of the radial diameter D2 of the outer occlusion structure 112, and the maximum radial diameter of the former is ¼ of the radial diameter D2 of the latter. Compared with the first embodiment, the outer occlusion structure 112 of this embodiment is more rounded and smoother at the distal end and can better conform to the shape of an intended aneurysm. Therefore, during release, it will exert a reduced impact on the dome of the aneurysm, lowering the risk of intraoperative bleeding. Furthermore, it exhibits enhanced axial support performance, which allows the device to better resist the “water-hammer” effect at an aneurysm neck opening over time during long-term occlusion, reducing the likelihood of recurrence of the aneurysm.

### Embodiment 3

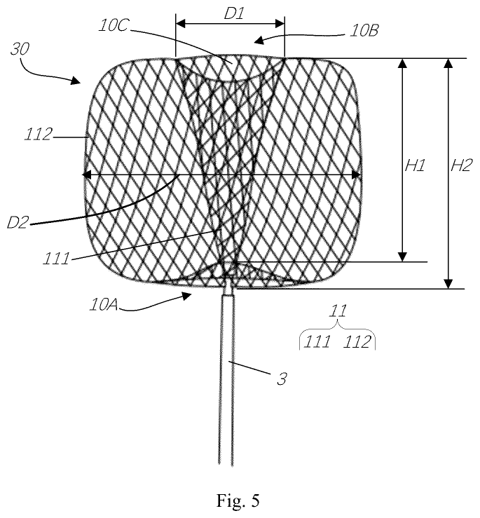

FIG. 5 shows an intratumoral occlusion device 30 according to a third embodiment of the present application. Corresponding structures in the intratumoral occlusion device 30 of this embodiment have been given the same reference numerals as those in the first embodiment. The intratumoral occlusion device 30 of this embodiment is structured substantially in the same way as the intratumoral occlusion device 10 of the first embodiment, and features common to the two embodiments are not repeated here. From the following description, those skilled in the art will appreciate how the third embodiment differs from the first embodiment.

In the exemplary embodiment of FIG. 5, in the expanded configuration, the outer occlusion structure 112 has a cylindrical outer contour and the inner support structure 111 has an inverted conical outer contour. Likewise, the inverted conical shape allows the intratumoral occlusion device 30 to encounter even less resistance during retrieval. That is, compared with the case of the fusiform inner support structure 111, the intratumoral occlusion device 30 will encounter less resistance during its retrieval into a microcatheter 1.

In this embodiment, the axial height H2 of the outer occlusion structure 112 that has a cylindrical outer contour is less than the radial diameter D2 in the expanded configuration. Accordingly, it can fill only part of the lumen of an aneurysm, but at the same time, can cover an aneurysm neck opening 21. Compared with the spherical outer occlusion structure 112, the cylindrical outer occlusion structure 112 has a flatter and larger proximal end face, which enables the device to better cover the aneurysm neck opening 21 and makes it suitable for use in the treatment of aneurysms with a wider neck.

Preferably, in the expanded configuration, the axial height H2 of the outer occlusion structure 112 is 0.6-0.8 times its radial diameter D2, to avoid the axial height H2 of the outer occlusion structure 112 being too small, which could affect stability of support.

As shown in FIG. 5, in one example, in the expanded configuration, the axial height H1 of the inner support structure 111 is ⅚ of the radial diameter D2 of the outer occlusion structure 112. This allows the outer occlusion structure 112 to be more rounded and smoother at the distal end and better conform to the shape of an intended aneurysm. Thus, the intratumoral occlusion device 30 will exert a reduced impact on the dome of the aneurysm during release, lowering the risk of intraoperative bleeding. Furthermore, it exhibits enhanced axial support performance, which allows the device to better resist the “water-hammer” effect at an opening of the aneurysm neck over time during long-term occlusion, reducing the likelihood of recurrence of the aneurysm.

In this embodiment, the maximum radial diameter of the inner support structure 111 is ⅓ of the radial diameter D2 of the outer occlusion structure 112, in the expanded configuration. This ensures both good axial support performance and a certain degree of compliance.

Finally, it is particularly noted that the outer contour of the expanded inner support structure 111 may also be of any other suitable shape than the fusiform or inverted conical shape described above, as long as the inner support structure 111 has a varying radial diameter D1. Preferably, the inner support structure 111 is broad in the middle and narrow at both ends, or broad at the distal end and narrow at the proximal end. The shape of the outer contour of the inner support structure 111 may be set by molding. Technically, this is relatively easy to implement. Likewise, the outer contour of the expanded outer occlusion structure 112 may also be of any other suitable shape than the spherical or cylindrical shape described above, as long as the proximal end face of the outer occlusion structure 112 can cover an aneurysm neck opening, while the rest can geometrically conform to the aneurysm wall to provide anchoring. Furthermore, when the outer contour of the outer occlusion structure 112 is cylindrical, the inner support structure 111 may also be fusiform in shape.

Compared with the prior art, the proposed intratumoral occlusion device is easier to release and less reliant on a surgeon's personal experience in aneurysm embolization and allows a surgical procedure using it to be finished within a shorter period of time. In addition, it has a single anchor at the proximal end, a shorter length after release and a compliant mesh at the distal end. All these can reduce its impact on the dome of an aneurysm, lowering the risk of aneurysm rupture. Moreover, the intratumoral occlusion device can be conformably filled in the lumen of an aneurysm through the outer occlusion structure, and a radial interference-fit can ensure the intratumoral occlusion device to be stably deployed in the lumen of an aneurysm. At the same time, the inner support structure can provide desirable axial support, and after occlusion of the neck of the aneurysm, long-term dimensional stability of the intratumoral occlusion device can be ensured, which reduces the axial shrinkage of the device and the likelihood of recurrence of the aneurysm. Additionally, the proximal end face of the outer occlusion structure can effectively occlude the neck of an aneurysm, facilitating healing of the aneurysm, in particular a wide-necked one. Furthermore, since the intratumoral occlusion device can be completely filled in an aneurysm, long-term dual antiplatelet therapy is not necessary, reducing the risk of hemorrhagic complications.

It should be noted that the embodiments disclosed herein are described in a progressive manner, with the description of each embodiment focusing on its differences from others. Cross reference can be made between the embodiments for their common or similar features. Since the system embodiments correspond to the method embodiments, they are described relatively briefly, and reference can be made to the method embodiments for more details thereof.

It also should be noted that while the invention has been described above with reference to preferred embodiments thereof, it is not limited to these embodiments. In light of the above teachings, any person familiar with the art may make many possible modifications and variations to the disclosed embodiments or adapt them into equivalent embodiments, without departing from the scope of the invention. Accordingly, it is intended that any and all simple variations, equivalent alternatives and modifications made to the foregoing embodiments based on the substantive disclosure of the invention without departing from the scope thereof fall within the scope.

Further, it is to be understood that, as used herein, the terms “first”, “second”, “third” and the like are only meant to distinguish various components, elements, steps, etc. from each other and are not intended to indicate logical or sequential orderings thereof, unless otherwise indicated or specified.

It is also recognized that the terminology used herein is for the purpose of describing particular embodiments only, and is not intended to limit the scope of the present invention. It must be noted that, as used herein and in the appended claims, the singular forms “a” and “an” include the plural reference unless the context clearly dictates otherwise. Thus, for example, a reference to “a step” or “a device” is a reference to one or more steps or devices and may include sub-steps and sub-devices. All conjunctions used are to be understood in the most inclusive sense possible. Thus, the term “or” should be understood as having the definition of a logical “or” rather than that of a logical “exclusive or” unless the context clearly necessitates otherwise. Further, implementation of the method and/or device according to the embodiments of the present invention may involve performing selected tasks manually, automatically, or a combination thereof.

1. An intratumoral occlusion device switchable between a collapsed configuration and an expanded configuration, comprising a mesh-like body structure and a proximal fixing structure, wherein the mesh-like body structure comprises an inner support structure and an outer occlusion structure, wherein the mesh-like body structure is made from a braided mesh tube, wherein the inner support structure is formed by inwardly folding one portion of the braided mesh tube into an interior of another portion of the braided mesh tube, wherein the outer occlusion structure is formed by the another portion of the braided mesh tube that is unfolded, wherein a proximal end of the outer occlusion structure is connected to the proximal fixing structure, and wherein a proximal end of the inner support structure extends toward and connected to the proximal fixing structure.

2. The intratumoral occlusion device of claim 1, wherein in the expanded configuration, a radial diameter of the inner support structure increases from a proximal end and a distal end to a middle portion of the inner support structure.

3. The intratumoral occlusion device of claim 1, wherein in the expanded configuration, a radial diameter of the inner support structure decreases from a distal end to a proximal end of the inner support structure.

4. The intratumoral occlusion device of claim 1, wherein in the expanded configuration, an outer contour of the inner support structure is a fusiform.

5. The intratumoral occlusion device of claim 1, wherein in the expanded configuration, an outer contour of the inner support structure is an inverted conical.

6. The intratumoral occlusion device of claim 4, wherein in the expanded configuration, a maximum radial diameter of the inner support structure is not greater than ½ of a radial diameter of the outer occlusion structure.

7. The intratumoral occlusion device of claim 1, wherein in the expanded configuration, an axial height of the outer occlusion structure is not greater than a radial diameter of the outer occlusion structure and not less than ½ of a radial diameter of the outer occlusion structure.

8. The intratumoral occlusion device of claim 7, wherein in the expanded configuration, the axial height of the outer occlusion structure is less than the radial diameter of the outer occlusion structure.

9. The intratumoral occlusion device of claim 7, wherein in the expanded configuration, an outer contour of the outer occlusion structure comprises a shape of a sphere or a cylinder.

10. The intratumoral occlusion device of claim 8, wherein in the expanded configuration, the axial height of the outer occlusion structure is 0.6-0.8 times the radial diameter of the outer occlusion structure.

11. The intratumoral occlusion device of claim 1, wherein in the expanded configuration: an axial height of the inner support structure is not greater than an axial height of the outer occlusion structure; and/or, the axial height of the inner support structure is not greater than a radial diameter of the outer occlusion structure.

12. The intratumoral occlusion device of claim 1, wherein in the expanded configuration: an axial height of the inner support structure is not less than ⅔ of an axial height of the outer occlusion structure; and/or, the axial height of the inner support structure is not less than ⅔ of a radial diameter of the outer occlusion structure.

13. The intratumoral occlusion device of claim 1, wherein mesh openings in a proximal end face of the outer occlusion structure have an area not exceeding 0.1 mm².

14. The intratumoral occlusion device of claim 1, comprising a proximal end and a distal end, wherein at the distal end of the intratumoral occlusion device, the braided mesh tube is inwardly folded and smoothly transits to form a distal opening.

15. The intratumoral occlusion device of claim 14, wherein in the expanded configuration, a diameter of the distal opening is not less than ⅓ of a radial diameter of the outer occlusion structure.
